(19) United States
(12) Patent Application Publication
(10) Pub. No.: US 2014/0166002 A1
(43) Pub. Date: Jun. 19, 2014

(54) JOINT OF SPUTUM SUCTION TUBE

(71) Applicant: PACIFIC HOSPITAL SUPPLY CO., LTD., Maoli County (TW)

(72) Inventors: Hsuan-Chiao HO, Miaoli County (TW); Ming-Chung CHEN, Miaoli County (TW)

(73) Assignee: PACIFIC HOSPITAL SUPPLY CO., LTD., Miaoli County (TW)

(21) Appl. No.: 13/716,151

(22) Filed: Dec. 16, 2012

(51) Int. Cl. A61M 16/04; A61M 1/00; A61M 16/20; A61M 16/00

(52) U.S. Cl. CPC A61M 16/0463; A61M 16/0057; A61M 1/0023; A61M 16/20
USPC 128/202.16

(57) ABSTRACT

A joint of sputum suction tube includes a patient tube (100), an exhaust tube (200) and a sleeve (300). The sleeve (300) includes a rotatable end (310) and a fixed end (320). The rotatable end (310) rotatably joints the exhaust tube (200). The fixed end (320) fixedly joints the exhaust tube (200). Therefore, the patient tube (100) rotatably connects with the exhaust tube (200) by the sleeve (300).

### BACKGROUND OF THE INVENTION

1. Field of the Invention

The present invention relates to a joint of sputum suction tube, and in particular to a joint of sputum suction tube with a patient tube and an exhaust tube which is rotatable corresponding to the patient tube.

2. Description of Related Art

Joints of sputum suction tube are used to suck secretion and sputum from the otolaryngology of a patient. The sputum suction tube is inserted in the otolaryngology of the patient. The outer end of the sputum suction tube is connected the joint of sputum suction tube. The joint of sputum suction tube could be applied for a variety of functions. Oxygen supplying and sputum suction are operated though the joint of sputum suction tube.

Moreover, the joint of sputum suction tube could be used for tube cleaning. Therefore, the joint of sputum suction tube has many manifolds which are provided for a variety of usages such as oxygen supplying and tube cleaning. It is inconvenient for user that the manifolds disturb each other.

### SUMMARY OF THE INVENTION

This invention provides a joint of sputum suction tube with a patient tube and an exhaust tube which is rotatable corresponding to the patient tube.

The joint of sputum suction tube includes a patient tube, an exhaust tube and a sleeve. The rotatable end rotatably joints the exhaust tube. The fixed end fixedly joints the exhaust tube. Therefore, the patient tube rotatably connects with the exhaust tube by the sleeve.

The rotatable end is inserted in the exhaust tube, and the fixed end is inserted in the patient tube. The joint of sputum suction tube further includes a fixing ring, a ring stopper is provided on the outer surface of the sleeve. The fixing ring is jointed to the exhaust tube and presses the ring stopper. The joint of sputum suction tube further includes a gasket, an annular trench is provided on the ring stopper, and the gasket is arranged in the trench.

The joint of sputum suction tube further includes a gasket hitched on the sleeve which is between the exhaust tube and the sleeve, an oxygen supplying manifold which is connected with the patient tube, an endoscope manifold which is connected with the patient tube, an injection manifold which is connected with the exhaust tube, and a rotary valve which is arranged on the exhaust tube and corresponded to the position of the injection manifold.

The present invention is a joint of sputum suction tube including a sleeve connecting with a patient tube and an exhaust tube, whereby the patient tube rotatably connect the exhaust tube. Thereof, the exhaust tube can be rotated depending on the operating condition of the joint of sputum suction tube to prevent from crossing of the appliances which are connected to the joint of sputum suction tube. Thus, the present invention amends the disadvantage of prior technologies.

### BRIEF DESCRIPTION OF DRAWING

FIG. 1 is a schematic view showing the first embodiment of the present invention;

FIG. 2 is a sectional view of an explosion diagram showing the first embodiment of the present invention;

FIG. 3 is an explosion diagram showing the first embodiment of the present invention;

FIG. 4 is a schematic view showing the second embodiment of the present invention;

FIG. 5 is an explosion diagram showing the second embodiment of the present invention.

### DETAILED DESCRIPTION OF THE INVENTION

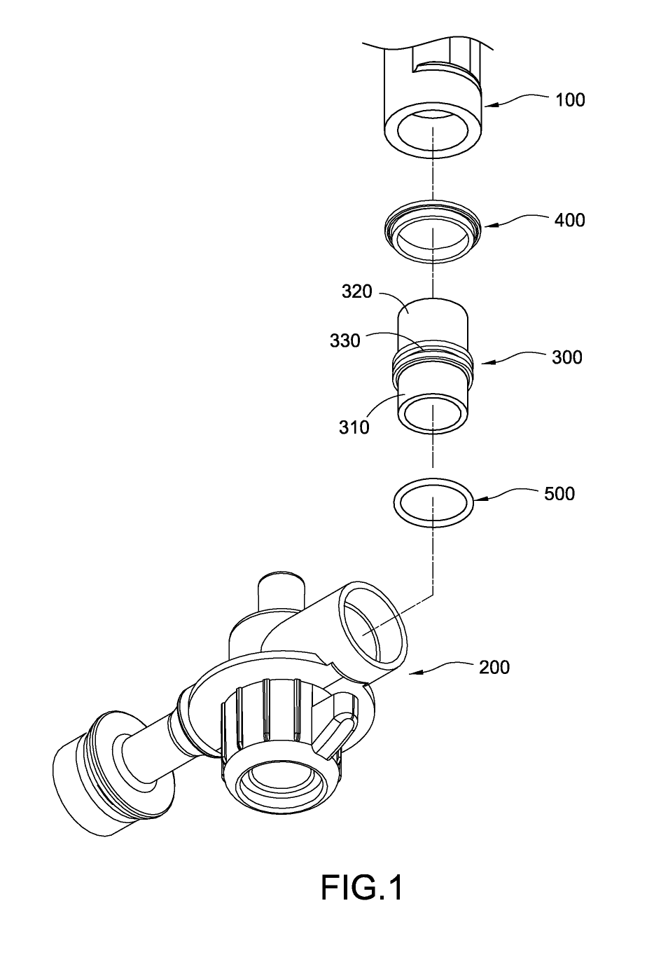

Please refer to FIG. 1. The first embodiment of the present invention suggests a joint of sputum suction tube. The joint of sputum suction tube includes a patient tube 100, an exhaust tube 200, a sleeve 300, a fixing ring 400 and a gasket 500. The sleeve 300 is used to rotatably connect the patient tube 100 with the exhaust tube 200.

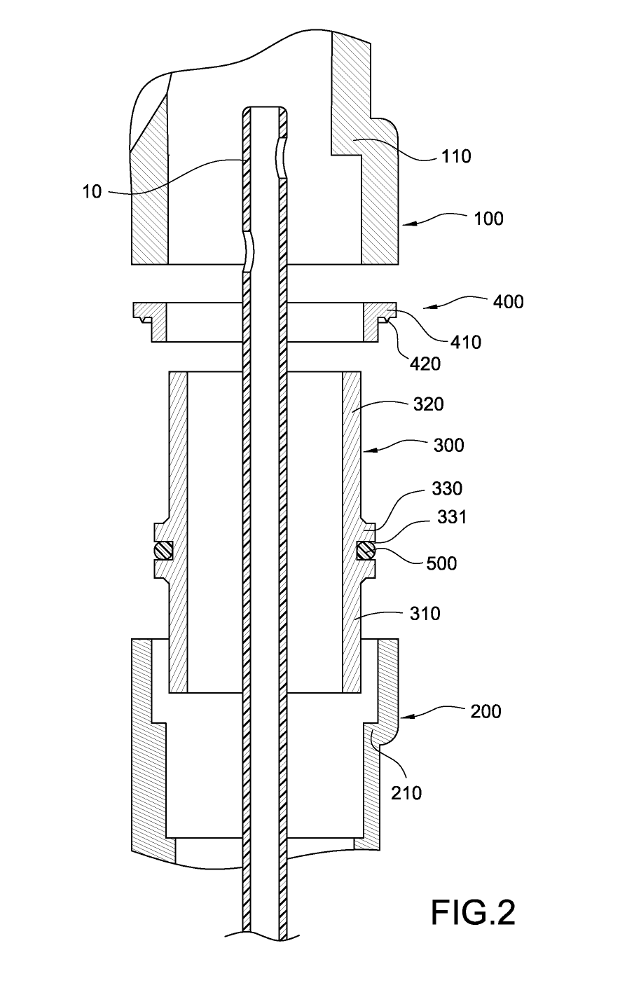

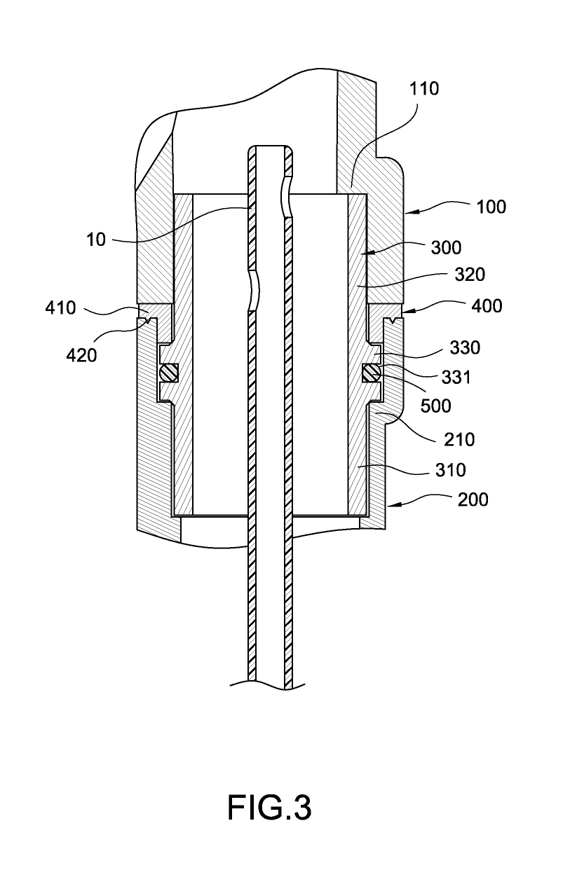

Please refer to FIGS. 1-3. The patient tube 100 is used to insert into an artificial otolaryngology which is set in a patient's body. The exhaust tube 200 is used to connect with a sputum suction tube. One end of the sputum suction tube is inserted into the artificial otolaryngology through the patient tube 100; the other end of the sputum suction tube is preferably connected with a vacuum to suck secretion and sputum from the otolaryngology of the patient.

The sleeve 300 includes a rotatable end 310 and a fixed end 320. The rotatable end 310 is rotatably inserted in the exhaust tube 200. The fixed end 320 is inserted in the patient tube 100 and fixedly connected with the patient tube 100. In the present embodiment, the fixed end 320 is interference fitted to the patient tube 100, but not limited thereto. Preferably, a stopper portion 110 is provided on the inner wall of the patient tube 100, the stopper portion 110 presses the edge of the opening of the fixed end 320 to fix the axial relative positions for the sleeve 300 and the patient tube 100. Preferably, a ring stopper 330 is provided on the outer surface of the rotatable end 310, and an annular trench 331 is provided on the ring stopper 330. The gasket 500, preferably arranged in the trench 331, is surrounded on the sleeve 300. Moreover, the gasket 500 is between the inner wall of the exhaust tube 200 and the outer wall of the sleeve 300. Therefore, the patient tube 100 and the exhaust tube 200 are sealing connected via the gasket 500.

The fixing ring 400 is arranged in the inner edge of the opening of the exhaust tube 200 and presses the ring stopper 330. Preferably, there is an annular rib 410 extended from the outer surface of the fixing ring 400. An annular ridge 420, pointing to the exhaust tube 200, is provided on the annular rib 410. The ridge 420 stabs into the edge of the opening of the exhaust tube 200, whereby the fixing ring 400 is fixed on the exhaust tube 200. The fixing ring 400 presses the ring stopper 330 to prevent the sleeve 300 from coming off the exhaust tube 200, and one end of the sleeve 300 is thereby rotatably jointed to the exhaust tube 200. The other end of the sleeve 300 is interference fitted to the patient tube 100. Therefore, the exhaust tube 200 is rotatably jointed to the patient tube 100 via the sleeve 300 and the fixing ring 400.

A flange 210, corresponding to the ring stopper 330, is preferably provided on the inner wall of the exhaust tube 200. The flange 210 presses the ring stopper 330, and the ring stopper 330 is caught between the flange 210 and the fixing ring 400. Therefore, the ring stopper 330 is unable to axially move corresponding to the exhaust tube 200.

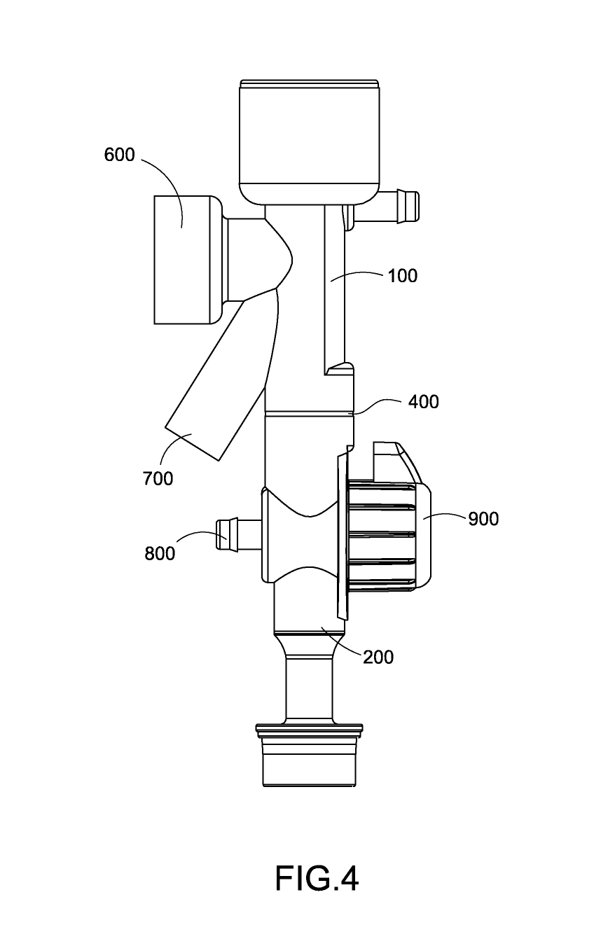

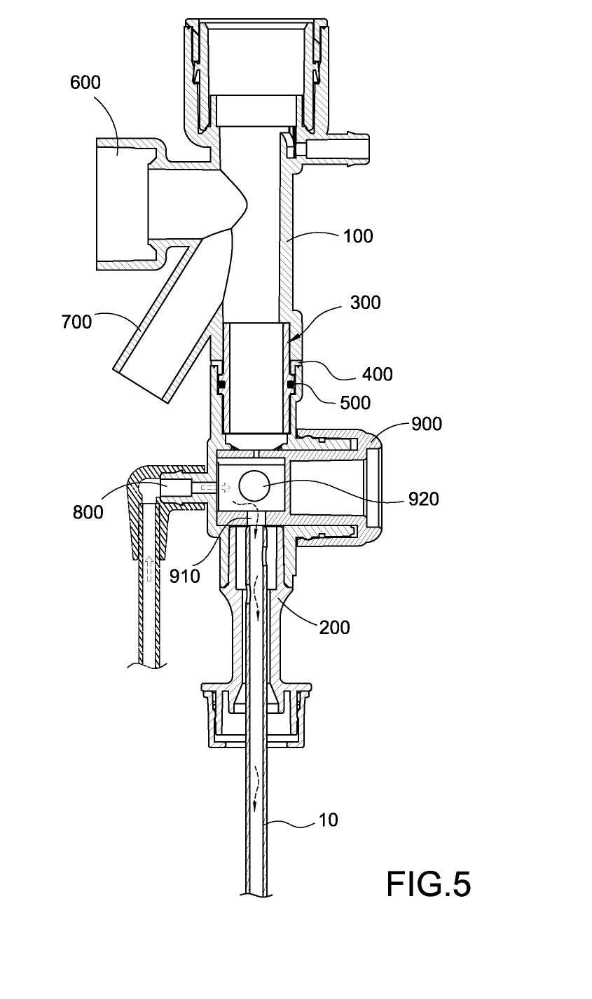

Please refer to FIGS. 4 and 5. The second embodiment of the present invention suggests a joint of sputum suction tube. The joint of sputum suction tube includes a patient tube 100, an exhaust tube 200, a sleeve 300, a fixing ring 400, a gasket 500, an oxygen supplying manifold 600, an endoscope manifold 700, an injection manifold 800, and a rotary valve 900. The sleeve 300 is used to rotatably connect the patient tube 100 with the exhaust tube 200. This embodiment is similar to the first embodiment, and the description for similar parts will be omitted below.

In this embodiment, the oxygen supplying manifold 600 is connected with the patient tube 100. The oxygen supplying manifold 600 is used to supply oxygen to the lung of patient through the oxygen supplying manifold 600 and the patient tube 100.

The endoscope manifold 700 is connected with the patient tube 100. The endoscope manifold 700 is used to contain a bronchoscope. The bronchoscope is inserted into the bronchus of the patient through the endoscope manifold 700 and the patient tube 100 in order to look over the bronchus.

The injection manifold 800 is connected with the exhaust tube 200 and preferably further connected with a rinsing pipeline to rinse the sputum suction tube 10. One end of the sputum suction tube 10 which is inserted in the patient could be pulled into the exhaust tube 200, thus some rinsing solution could be injected into the exhaust tube 200 through the injection manifold 800 to rinse the sputum suction tube 10. Further, the wasted rinsing solution is sucked out through the sputum suction tube 10 via the vacuum which is connected with the sputum suction tube 10.

Please refer to FIG. 5. The rotary valve 900 is provided on the exhaust tube 200 and corresponding to the position of the injection manifold 800. A drainage hole 910 and an insertion hole 920, toward a variety of directions, are provided on the rotary valve 900. While the sputum suction tube 10 is rinsed, the rotary valve 900 is rotated to make the insertion hole 920 be covered and closed by the inner wall of the exhaust tube 200. Meanwhile, the drainage hole 910 is aligned with the sputum suction tube 10, and the exhaust tube 200 and the patient tube 100 are separated by the rotary valve 900. The rinsing solution is injected into the rotary valve 900 through the injection manifold 800, and then flows into the sputum suction tube 10 through the drainage hole 910. The rotary valve 900 could prevent the rinsing solution from flowing toward the patient tube 100. In a general operating condition, the sputum suction tube 10 should be inserted into the patient through the exhaust tube 200 and the patient's otolaryngology tube 100. Meanwhile, the rotary valve 900 could be rotated to allowed the sputum suction tube 10 pass the insertion hole 920 and further through the rotary valve 900, and the drainage hole 910 is preferably covered and closed by the inner wall of the exhaust tube 200.

The joint of sputum suction tube of present invention provides the sleeve 300 which is connected between a patient tube 100 and an exhaust tube 200, whereby the patient tube 100 is rotatably jointed to the exhaust tube 200. Therefore, the exhaust tube 200 could be rotated to adjust its position depending on the operating condition joint of sputum suction tube. The present invention could prevent the appliances which are connected to the joint of sputum suction tube from crossing of. Thus, the present invention amends the disadvantage of prior technologies.

Although the present invention has been described with reference to the foregoing preferred embodiment, it will be understood that the invention is not limited to the details thereof. Various equivalent variations and modifications can still occur to those skilled in this art in view of the teachings of the present invention. Thus, all such variations and equivalent modifications are also embraced within the scope of the invention as defined in the appended claims.

What is claimed is:

1. A joint of sputum suction tube, comprising:
a patient tube (100);
an exhaust tube (200); and
a sleeve (300), comprising a rotatable end (310) and a fixed end (320), the rotatable end (310) being rotatably jointed to the exhaust tube (200), the fixed end (320) being fixedly jointed to the patient tube (100).

2. The joint of sputum suction tube according to claim 1, wherein the rotatable end (310) is inserted in the exhaust tube (200).

3. The joint of sputum suction tube according to claim 1, wherein the fixed end (320) is inserted in the patient tube (100).

4. The joint of sputum suction tube according to claim 1, further comprising a fixing ring (400), a ring stopper (330) being provided on an outer surface of the sleeve (300), and the fixing ring (400) being connected with exhaust tube (200) and pressing the ring stopper (330).

5. The joint of sputum suction tube according to claim 4, further comprising a gasket (500), an annular trench (331) being provided on the ring stopper (330), and the gasket (500) being arranged in the trench (331).

6. The joint of sputum suction tube according to claim 1, further comprising a gasket (500), the sleeve (300) being hitched the gasket (500) which is between the exhaust tube (200) and the sleeve (300).

7. The joint of sputum suction tube according to claim 1, further comprising an oxygen supplying manifold (600), the oxygen supplying manifold (600) being connected with the patient tube (100).

8. The joint of sputum suction tube according to claim 1, further comprising an endoscope manifold (700), the endoscope manifold (700) being connected with the patient tube (100).

9. The joint of sputum suction tube according to claim 1, further comprising an injection manifold (800), the injection manifold (800) being connected with the exhaust tube (200).

10. The joint of sputum suction tube according to claim 7, further comprising an endoscope manifold (700), the endoscope manifold (700) being connected with the patient tube (100).

11. The joint of sputum suction tube according to claim 7, further comprising an injection manifold (800), the injection manifold (800) being connected with the exhaust tube (200).

12. The joint of sputum suction tube according to claim 8, further comprising an injection manifold (800), the injection manifold (800) being connected with the exhaust tube (200).

13. The joint of sputum suction tube according to claim 12, further comprising an oxygen supplying manifold (600), the oxygen supplying manifold (600) being connected with the patient tube (100).

14. The joint of sputum suction tube according to claim 9, further comprising a rotary valve (900), the rotary valve (900) being arranged on the exhaust tube (200) and corresponded to a position of the injection manifold (800).

15. The joint of sputum suction tube according to claim 11, further comprising a rotary valve (900), the rotary valve (900) being arranged on the exhaust tube (200) and corresponded to a position of the injection manifold (800).

16. The joint of sputum suction tube according to claim 12, further comprising a rotary valve (900), the rotary valve (900) being arranged on the exhaust tube (200) and corresponded to a position of the injection manifold (800).

17. The joint of sputum suction tube according to claim 13, further comprising a rotary valve (900), the rotary valve (900) being arranged on the exhaust tube (200) and corresponded to a position of the injection manifold (800).
